(19) United States
(12) Patent Application Publication
(10) Pub. No.: US 2008/0119774 A1
(43) Pub. Date: May 22, 2008

(54) Bandaging structure and methodology

(75) Inventor: Gerhard Paasche, Scappoose, OR (US)

Correspondence Address: ROBERT D. VARITZ, P.C., 4915 SE 33RD PLACE, PORTLAND, OR, 97202, US

(73) Assignee: MJD Innovations, L.L.C.

(21) Appl. No.: 11/983,259

(22) Filed: Nov. 7, 2007

Related U.S. Application Data
(60) Provisional application No. 60/859,770, filed on Nov. 16, 2006.

(51) Int. Cl. A61F 13/00

(52) U.S. Cl. 602/53

(57) ABSTRACT

Wound bandaging/dressing foam structure including a low rebound, temperature-conforming, acceleration-rate-sensitive cushioning expanse applicable to wound-proximate-associated anatomical tissue to apply a predetermined expanse of substantially uniform, non-blood-flow-occluding pressure, accompanied by adjacent, included lateral extremities of this expanse which apply a gradually declining-to-zero anatomical-tissue pressure.

### RELATED APPLICATIONS

This patent application claims priority from U.S. Provisional Patent Application Ser. No. 60/859,770, for Bandaging Structure and Methodology, filed Nov. 16, 2006, which describes improvements and variations to the subject matter disclosed, illustrated and claimed in U.S. Pat. No. 6,812,375 B2, granted Nov. 2, 2004, for Pressure-evenizing Low-rebound Wound Dressing, the contents of the provisional patent application and the patent are incorporated herein by reference.

### FIELD OF THE INVENTION

This invention relates to medical dressing for wounds, and specifically to a medical dressing which provides evenly distributed pressure about a wound site.

### BACKGROUND OF THE INVENTION

A background description of the prior art is found in U.S. Pat. No. 6,812,375 B2. This application describes further improvements and modifications to the subject matter of the '375 patent. One improvement is the incorporation into an otherwise generally uniform-thickness, pressure-evenizing foam structure of surrounding, lateral wane regions, which diminish in thickness to zero. These wane regions create a “downward” wound-area-applied pressure gradient adjacent lateral regions of a wound, with appropriate use of a dressing formed in accordance with the invention.

Two other improvements include (a) the utilization of a relatively rigid shape-holding, and generally bandaging-three-dimensional shape-defining, shell which is fitted with pressure-applying foam in accordance with the invention, and (b), a clamshell-type arrangement in which a clamshell style, generally rigid shell structure holds a pair of pressure-applying foam structures to enable complete wrap-around, or substantially full-enclosure, dressing of a wound which exists in an extremity, such as in a hand or a foot.

By way of additional background, the conventional way of post operational bandaging, for example, in the case of a knee replacement, requires, after stapling the skin together as the final step of the operation, application of a sterile fleece, which serves for the collection of blood coming out of the wound. Additional cloth padding that is intended to add pressure to the operated area to compress the tissue, to suppress internal bleeding, and to suppress formation of cavities that may fill with blood and other body fluids, is placed on top of the sterile fleece. As the final step, a five inch wide elastic bandaging is wrapped in a spiral around the leg, beginning at foot, with overlapping, and terminating at the top of the thigh.

Uneven pressure distribution and local stagnation lead to the suppression of flow in the venous and lymphatic systems, increasing the risk of thrombosis and other problems related to circulation. These are evidenced by visible pressure lines, which may be observed with removal of the elastic bandage.

Occasionally, especially with post-operative restless patients, the bandage may be displaced, and may, on occasion, become totally unwrapped, which requires that the elastic bandage be repositioned, with further implied complications. This is inconvenient, especially as the time-consuming bandaging unnecessarily occupies nursing personnel, preventing their attention to other duties.

If the bandaging is not carefully applied, the likelihood of ulcers forming e.g., on the heel, may increase, which adds further complications, such as extended heeling time and complicated patient rehabilitation.

Immediately after the operation, continuous passive motion (CPM) is used with a guide apparatus. Conventional bandaging results in pinching of the bandaging on the back of the knee, resulting in generally vocal complaints from the patients, thus limiting willing patient cooperation.

### SUMMARY OF THE INVENTION

Wound bandaging/dressing foam structure including a low rebound, temperature-conforming, acceleration-rate-sensitive cushioning expanse applicable to wound-proximate-associated anatomical tissue to apply a predetermined expanse of substantially uniform, non-blood-flow-occluding pressure, accompanied by adjacent, included lateral extremities of this expanse which apply a gradually declining-to-zero anatomical-tissue pressure.

It is an object of the invention to provide a bandaging structure having a generally uniform-thickness, pressure-evenizing foam structure of surrounding, lateral wane regions which diminish in thickness to zero.

Another object of the invention is to provide a relatively rigid shape-holding, and generally bandaging-three-dimensional shape-defining, shell which is fitted with pressure-applying foam.

Yet another object of the invention is to provide a clamshell-type arrangement in which a clamshell style, generally rigid shell structure holds a pair of pressure-applying foam structures to enable complete wrap-around, or substantially full-enclosure, dressing of a wound which exists in an extremity, such as in a hand or a foot.

This summary and objectives of the invention are provided to enable quick comprehension of the nature of the invention. A more thorough understanding of the invention may be obtained by reference to the following detailed description of the preferred embodiment of the invention in connection with the drawings.

### BRIEF DESCRIPTION OF THE DRAWINGS

FIG. 1 is a perspective representation of a first embodiment of a pressure pad of the invention.

FIG. 2 is a cross-section of the pressure pad of FIG. 1.

FIG. 3 is a perspective representation of a second embodiment of the pressure pad of the invention.

FIG. 4 is a cross-section of the pressure pad of FIG. 3.

FIG. 5 is a section through a wound site with a pressure pad of the invention applied thereto.

FIG. 6 depicts a pressure pad of the invention as used for applying pressure to an appendage.

FIG. 7 depicts a tourniquet application of the pressure pad of the invention.

### DETAILED DESCRIPTION OF THE PREFERRED EMBODIMENTS

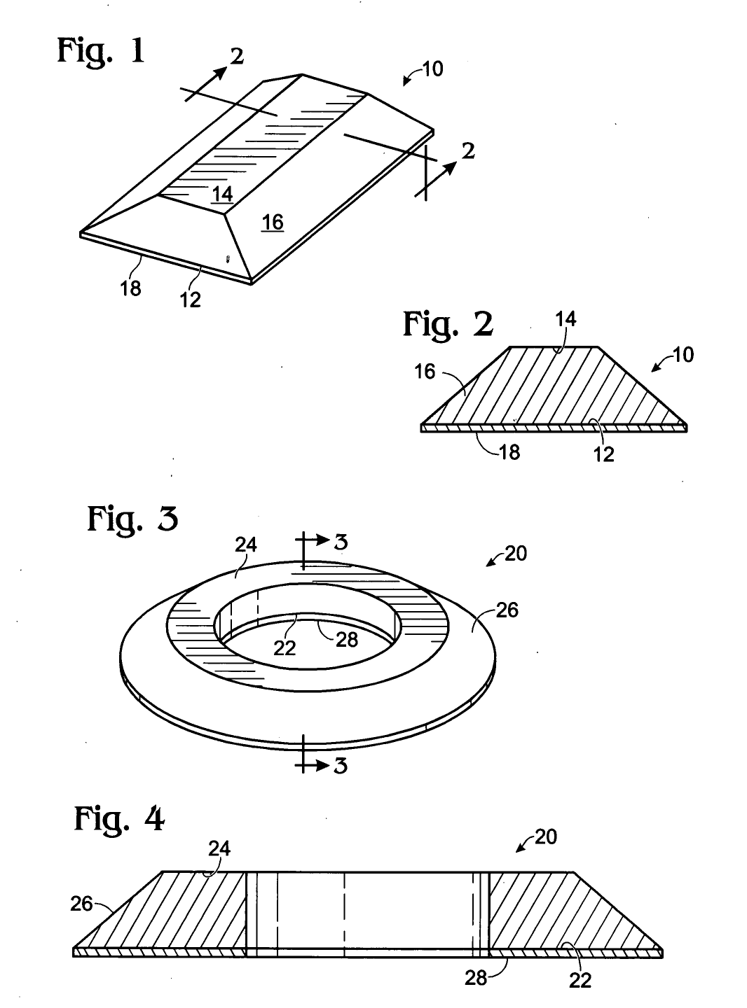

Referring now to FIGS. 1 and 2, a first embodiment of the pressure pad structure of the invention is depicted generally at 10. As noted in the above-identified patent, pressure pad structure 10 is formed of a material known as Confor, which is made by EAR Specialty Composites of Indianapolis, Ind. The specific material, in the preferred embodiment, is Confor CF-40, although other Confor materials may be used, with the other materials having durometer ratings different than CF-40. The material used in pad 10 is a compressible, low rebound, acceleration-rate-sensitive material which, after a compression deformation, exhibits a slow creep return toward an undeformed condition. Any foam material exhibiting the foregoing characteristics may be used as the visco-elastic foam material in the practice of the invention or of the method of the invention. Pad 10 includes a lower surface 12, which is directed towards the patient in use, an upper surface 14, and a peripheral expanse, which tapers from upper surface 12 to lower surface 14 in a wane region 16.

Joined to that face of the lower surface is a thin layer 18, typically about 0.003- to about 0.01-index, of a suitable moisture-barrier material which is made herein of a sprayed-on layer formed of a vinyl-solvent-based material known as Russell Coating, sold under the product designator V-2000, and manufactured by Russell Products Company, Inc., of Akron, Ohio. In the embodiment of the invention now being described, it is the exposed facial expanse specifically of moisture-barrier layer 18 which substantially directly engages, in most instances, the surface area of the wound area to which bandaging pressure is applied by device 10. As will become evident after reading the entire Specification hereof, moisture-barrier layer 18 may be fixed to pad 10, or may be applied directly to a patient, or over a fleece absorbance layer, thus, pad 10 may be formed with or without integral moisture-barrier layer 18.

Thus, pad 10 provides a wound bandaging/dressing foam structure having a low rebound, temperature-conforming, acceleration-rate-sensitive cushioning expanse applicable to wound-proximate-associated anatomical tissue to apply a predetermined expanse of substantially uniform, non-blood-flow-occluding pressure, accompanied by adjacent, included lateral extremities of this expanse which apply a gradually declining-to-zero anatomical-tissue pressure. The cushioning expanse includes a central body of one relatively uniform thickness having lateral-edge wane regions tapering from such relatively uniform expanse thickness to zero thickness.

The width, height and length of pressure pad structure are all variable. Base shapes can be triangle circular or any odd shape. The beveling of the edges creates a pressure transition area and with that, avoids pressure lines at the edges of the pressure pad. It also allows for overlapping of pieces to branch out, as applicable in a phlebectomy. It has been shown that when stripping out the veins during a phlebectomy, blood pooling and blisters usually associated with this operation can be almost totally prevented. This naturally leads to a very short healing time and also to fewer complications, and is thus specifically well suited for use in such a procedure.

The cure of decubitus ulcers (bedsores) is another application which warrants special attention, as it has to do with a careful balance of pressures around the wound areas. The need to apply a local pressure that restricts blood and lymph flow laterally (sideways) in the surface areas towards the wound, and may allow the body to feed in the needed support for building new tissue vertically from below. Referring now to FIGS. 3 and 4, an embodiment of the pressure pad structure of the invention is depicted generally at 20, which structure is useful to speed recovery of ulcers. Pad 20 is formed much like pad 10, having an a lower surface 22, which is directed towards the patient in use, an upper surface 24, and a peripheral expanse, which tapers from upper surface 22 to lower surface 24 in wane expanse region 26. A layer of a suitable moisture-barrier material 28 is formed on lower surface 24.

Pressure pad 20 may be placed around an ulcer, and when held in place by an elastic bandage, the foam material will reduce the blood pressure in the surrounding tissue, thus allowing for the healing of the ulcer. The same technique may be applied to ulcers in the area of the head, by using a rounded shell, similar to a helmet, which is padded on the inside with the visco elastic foam. This distributes the weight of the head to different areas and allow healing of the ulcer. In addition, it is relatively comfortable, resulting in acceptance by patients. This helmet kind of a device may be made of a simple vacuum molded shell which is strapped to the head of the patient, or if preferred, is integrated into a “pillow style” cushion. As there is generally little movement associated with the presence of ulcers, ventilation must be provided to the areas around the wound, which ventilation may be provided by varying the shape and size of the ring. Beveling is important to the outside to have a smooth transition to the pressure zone, e.g., parallel surface area.

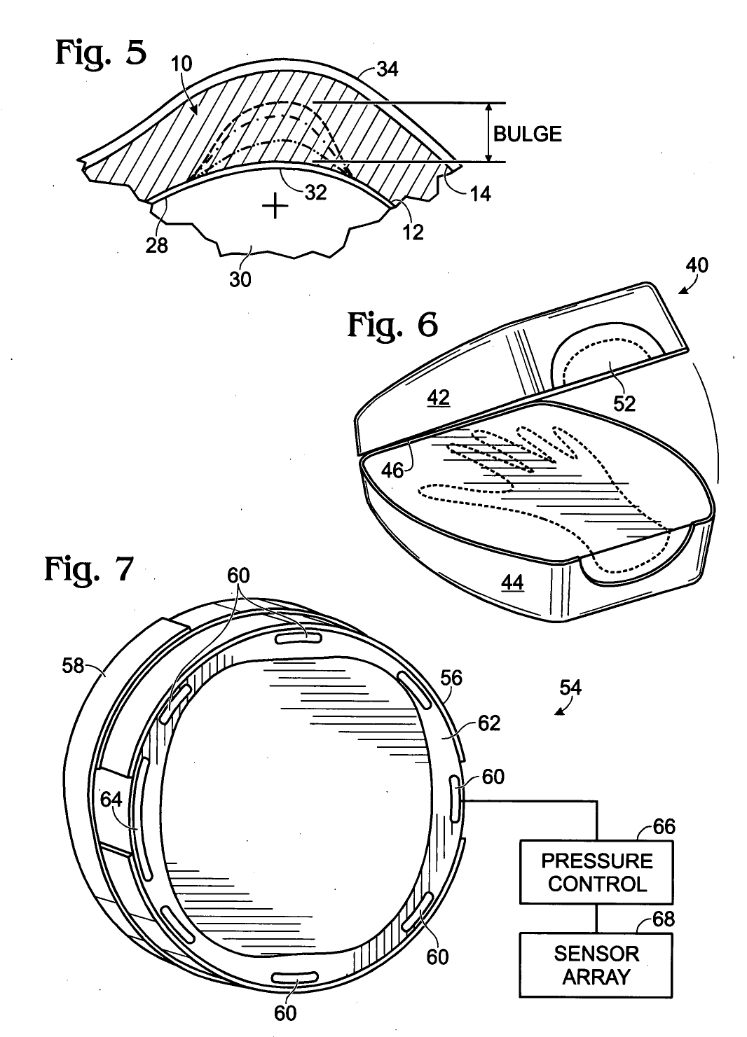

Referring now to FIG. 5, pressure pad 10 is depicted in situ, as applied to an extremity 30. The region of the anatomy which is bandaged by pressure pad 10 is generally shown at 30, and is here pictured with a quite uneven, undulating topography. Specifically also illustrated in FIG. 5, and underlying the surface of this anatomical site as is indicated by a cross (+) is a pulsatile blood-flow artery or vein. This region of the anatomy is especially distortedly emphasized in FIG. 5, in order aid in explaining and visualizing an important behavioral operation of the invention. An exaggerated BULGE is shown and so labeled in FIG. 5.

In dashed lines in FIG. 5, this particular area of site 30 in the anatomy is shown distended, or bulged, upwardly to follow a line which is pictured by various styles of dashed lines. This region, under such a circumstance, is clearly not a candidate for the conventional application of overly high pressure in a normal bandaging arrangement.

Uniquely, and because of the acceleration-rate-sensitive nature of pad 10, when it is appropriately placed to apply pressure to wound area 32, the skin-facing surface region of pad 10 and layer 18 adjusts its topographical characteristics so as to produce, ideally, a substantially matching complementary topography—and very specifically a matching topography which tends to produce a generally evenized pressure over the entirety of site 32.

Thus, from a purely static pressure-applying point of view, the portion of device 10 which acts through surface 12 and layer 18, directly over wound site 32 tends to adjust so as to apply appropriate topographically-following, relatively even pressure such that undesirable overpressure and underpressure conditions mentioned above do not come into play.

From a dynamic point of view, and in response to pulsatile behavior as pictured in FIG. 5, the acceleration-rate-sensitive material in pad 10 tends to respond to increased bulging, as a consequence of periodic blood flow, to yield, and when bulging begins to recede, to follow that receding activity with little or no appreciable spring-rate behavior. A consequence of this is that, even in a dynamic pulsatile region in a bandaging site, the pad of this invention tends to adapt appropriately and easily to changing topographic conditions, and specifically in a manner tending to maintain substantially even overall bandaging pressure. The moisture-barrier layer 18 prevents the invasion of any weeping fluid near the wound site into pad 10.

In practice, following knee-joint replacement, the complete leg, including the foot, was wrapped in foam and kept it in place with elastic band wrapping as normally used for bandaging, to apply pressure after the operation. As a result of the use of the pad and method of the invention, the leg and foot looked well two-days after the operation when the bandaging was removed.

It will be understood by one of ordinary skill in the art that the moisture-barrier layer integrally formed with the pad prevents the dissipation of moisture from the wound, whether such moisture is the result of perspiration or seepage from the wound. Regardless of the source, moisture needs to be removed from the vicinity of the wound. As a difference to the usual practice of just using a layer of gauze to collect the bleeding out of the wound during the first days after the operation, a layer of a sterile cellophane-like membrane was placed on top of the wound area to avoid any contamination which might come from the foam. This is essentially moving the moisture-barrier layer from the foam to the leg as a self-adhesive layer. Another alternative for solving the problem of moisture transport away from the tissue under the moisture-barrier layer is to use a sweat wicking fabric to enhance the wicking of sweat that gets transpired by the skin.

Referring now to FIG. 6, the foam material used for pressure pads 10 and 20 is encapsulated in a shell enclosure 40 that is essentially a two-sided shell style enclosure having a top side 42, a bottom side 44, which sides are held together by a hinge 46, and which further has an opening 48 on the non-hinged side which allows a foot or hand to be transitioning to the inside providing even pressure to the foot/hand after an operation with preformed foam inserts 50, 52, which may have a moisture-barrier layer or not. The foam inserts may be replaced when the shell is next used. Easy access for control after the operation and during the healing process is provided through hook and loop fasteners (not shown).

Referring to FIG. 7, a portion of another embodiment of the invention is depicted generally at 54, and is similar to a pneumatic tourniquet, or blood pressure measuring bladder, except that it is sized to surround or cover a complete extremity, forming a sleeve thereover, and includes multi-part chambers, which take the form of a series of rings extending about the interior of a banding structure, or which may extend at various angles from the banding structure. A shell 56, formed in sections, extends the length of the sleeve 54. Shell 56 may be formed of a rigid or semi-rigid material. Alternately, a flexible material may be used so long as such material has an unyielding outer skin, so that pressure may be applied to the desired location.

This sock or bladder style configuration, which is strapped around the extremity or body part, and held in place by hook-and-loop fasteners 58, has an integrated array of air chambers 60, part of a pressurization mechanism, on the inside thereof, which applies pressure to foam 62 to achieve even distribution and breathing activity. A cooling mechanism is provided and includes vessels 64 which allow a coolant to circulate throughout sleeve 54, is located between the foam and the bladder (air chambers), which allows flexibility for exercising and, if needed, may be opened and closed at any time for inspection. While bladder tourniquets are known, none have the visco-elastic foam described herein generating the transitional evenizing pressure of the invention. A pressure control 66 is provided which adjusts the pressure depending on the orientation of the patient, to compensation for blood pressure variations, and to provide application of pressure to control a swelling of e.g., a leg, which orientation, and other parameters, is detected by a sensor array 68, which comprise the remainder of the pressurization mechanism. The continuous pressure control automatically adjusts as the swelling becomes less, while the visco-elastic foam maintains constant pressure. Other features of such an active system allow application of pressure curve profiles, together with temperature profile curves, for therapeutic reasons and to allow to release of some pressure for a limited amount of time, or as well allowing the patient to adjust pressure to a comfortable level, while not dropping below a predetermined minimum set by the attending physician. As the foam layer is interchangeable, foams of various durometer ratings may be used for different applications, pressure ranges and temperature ranges. Experience has shown that the distal end of the extremity should be exposed to the same amount of outside pressure as is the proximal end, otherwise any unpressurized areas on the distal side of the bandaging is likely to swell, which likely will lead to additional complications.

The foam attached to the inside of variously formed shells, that may be either fairly rigid or flexible, depending on the position of the application as well as the complexity of the underlying shape that needs to be covered. Variations in the foam durometer may be used to detect especially sensitive areas and to control the amount of pressure. The shell shapes may be as simple as a flat piece of plastic bent into a channel form up to fairly complicated shapes that may be either vacuum formed plastics or other preshaped formed materials with surfaces that have the foam applied to the inside.

In actual patient applications, the complete leg, including the foot, was wrapped in foam, kept in place with an elastic band wrapping, as normally used for bandaging, to apply pressure after the operation. As a result, the leg and foot looked well after the operation when the bandaging was removed two days after the operation. As a difference to the usual practice of just using a layer of gauze to collect the bleeding out of the wound during the first days after the operation, a layer of a sterile cellophane-like membrane was applied to the top of the area to avoid contamination which might originate in the foam. This is essentially moving the coating from the foam to the leg as a self adhesive layer that is available and also sterile.

### EXAMPLE 1

A knee replacement operation was performed on a patient. Such an operation is a severe intrusion into the body, in which the complete knee joint is replaced by an artificial joint. The access cut that is needed to perform this surgery is about 10 inches long. There is a significant amount of tissue separation involved in this operation as well as other traumatic events taking place at the same time, e.g., to the circulatory system. Fleece was applied to the wound, which was covered by a pressure pad of the invention, and the leg wrapped with an elastic bandage. After two days, a comparison of a patient who was bandaged using conventional techniques was compared to the patient bandaged using the pressure pad structure and methodology of the invention. The evaluation by the attending physician, who had performed the operations, was that there were significant differences between the two patients: (1) when opening the bandaging with the added foam two days after the operation, there was significantly less contamination of blood in the fleece on the wound compared to the conventionally bandaged patient; (2) the appearance of the skin and overall appearance of the leg tissue of the patient bandaged using the pressure pad and method of the invention was very healthy; and (3) overall swelling of the leg was less.

The second patient, having received conventional bandaging applied after the operation, had significant signs of suppression of circulation to the surface of the skin, a common cause of additional complications, there was also more bleeding into the fleece, which had come out of the actual surgical cut. Overall, the conventionally bandaged patient exhibited indications that the applied pressure after the operation was not as uniform as in the other patient.

### EXAMPLE 2

A phlebectomy procedure, the removal of varicose veins, was treated post-operatively using the pressure pad and method of the invention. A sterile fleece provided a catch for blood coming out of the wound. A sterile, self-adhesive foil was applied to fix the fleece in place. A cotton sock, as commonly used with a plaster-cast, was used to cover the leg top-to-bottom as a separation layer under the foam. Then the foam was applied over the affected area of the leg and elastic bandaging applied on top of the foam to provide the needed pressure.

The comfort for the patient was very good. The pressure pad and method of the invention proved to be very useful, especially when using the CPM system, which is a critical part of rehabilitation following joint replacement, to allow for exercising the joint without muscle flexion/extension by the patient. The compression of the wounded area as well as the rest of the leg was very good. The tissue texture (appearance) for the areas covered with the foam was excellent and indicated that there were no complications to be expected, as might usually appear. The forming of ulcers using the pressure pad and method of the invention is virtually impossible. This is an important consideration, especially for older patients who may have very thin and sensitive skin.

Thus, a pressure pad structure and method of use for post-operative wound dressing has been disclosed. It will be appreciated that further variations and modifications thereof may be made within the scope of the invention as defined in the appended claims.

I claim:

1. Wound bandaging/dressing foam structure including a low rebound, temperature-conforming, acceleration-rate-sensitive cushioning expanse applicable to wound-proximate-associated anatomical tissue to apply a predetermined expanse of substantially uniform, non-blood-flow-occluding pressure, accompanied by adjacent, included lateral extremities of this expanse which apply a gradually declining-to-zero anatomical-tissue pressure.

2. The structure of claim 1, wherein the cushioning expanse includes a central body of one relatively uniform thickness having lateral-edge wane regions tapering from such relatively uniform expanse thickness to zero thickness.

3. The structure of claim 2 which further includes a generally shape-defining, relatively rigid shell supporting an outer side of the foam structure.

4. The structure of claim 1 which further includes a generally shape-defining, relatively rigid shell supporting an outer side of the foam structure.

5. A wound bandaging/dressing foam structure including a low rebound, temperature-conforming, acceleration-rate-sensitive cushioning expanse organized in a shell to envelop a wound in an envelopable anatomical extremity.

6. The structure of claim 5 which includes a pressurization mechanism therein, having bladders located between said cushioning expanse and said shell.

7. The structure of claim 5 which includes a cooling mechanism therefore, wherein the cooling mechanism includes cooling vessels extending throughout said shell.

8. The structure of claim 5 wherein said shell is in the form of a clam shell.

9. The structure of claim 5 wherein said shell extends the full length of an extremity.

10. The structure of claim 5 wherein said shell extends less than the full length of an extremity.
